(19) United States
(12) Patent Application Publication
(10) Pub. No.: US 2022/0328147 A1
(43) Pub. Date: Oct. 13, 2022

(54) SYSTEMS AND METHODS FOR DETECTING DRUG-DISEASE STATE INTERACTIONS IN PATIENTS

(71) Applicants: Christine Andrea WRIGHT, Lakeland, FL (US); Leslie Renee Cadet, Rancho Cucamonga, CA (US)

(72) Inventors: Christine Andrea WRIGHT, Lakeland, FL (US); Leslie Renee Cadet, Rancho Cucamonga, CA (US)

(21) Appl. No.: 17/708,366

(22) Filed: Mar. 30, 2022

Related U.S. Application Data
(60) Provisional application No. 63/173,101, filed on Apr. 9, 2021.

(51) Int. Cl. G16H 10/60; G16H 20/10; G16H 50/20; H04L 9/08

(52) U.S. Cl. CPC G16H 10/60; G16H 20/10; G16H 50/20; H04L 9/088

(57) ABSTRACT

A drug-disease interaction detection server determines a presence or absence of contraindications or relevant precautionary conditions for a treatment for a patient by parsing database(s) based on the treatment, determines values of factors corresponding to the patient based on a real-time analysis of data obtained from an EMR system, generates a total score by computing a total sum of at least a subset of the values of factors, determines whether the total score is greater than or less than or equal to a predetermined threshold, generates a recommendation for prescribing the treatment requested for the patient in response to the determination of the total score and an indication being treated in the patient, determines an availability or unavailability of one or more alternative treatments, generates an additional recommendation for the patient, and receives an order for a selected treatment for the patient based on the additional recommendation.

### CROSS-REFERENCE TO RELATED APPLICATIONS

This application claims the benefit under 35 U.S.C. § 119(e) of U.S. Provisional Patent Application No. 63/173,101, filed April 9, 2021, which is incorporated by reference herein in its entirety.

### BACKGROUND

### Field

The present disclosure relates to real-time systems and methods for dynamically detecting drug-disease state interactions in patients and providing recommendations to healthcare providers to help improve patient safety and reduce medication errors as adverse drug events are the fourth leading cause of death, and in many cases can be prevented.

### Background

Healthcare providers are often given only a limited amount of time to manage the care of many patients within a given day, in which each patient may have varying health conditions and associated prescriptions for each condition. Patients may see several different specialists and healthcare providers in addition to their primary care physicians, each of whom may prescribe different drugs to treat a plethora of health conditions in each patient, thereby increasing the patient's risk for developing adverse drug events. In some cases, healthcare providers, such as physicians, nurse practitioners, physician assistants, clinical pharmacists, specialists, and/or the like, may utilize various electronic medical record (EMR) systems to access patient data that informs providers of a wealth of information concerning the patient, i.e. their present health conditions, current medications prescribed, lab data, and adverse drug reaction history.

Conventional EMR systems are limited in that they do not provide information regarding drug-disease interactions to healthcare providers for patients. Drug-disease interactions indicate circumstances where the pharmacotherapy used to treat a disease causes worsening of another disease in a patient. In some cases, prescribing certain drugs may increase risks for an adverse reaction in a patient with one or more underlying conditions or diseases. In such circumstances, certain drugs should be avoided, adjusted, or prescribed with caution and monitored in patients with specific disease states.

Such considerations, however, may be missed during routine follow-up appointments with patients for disease state management, as EMR systems used by healthcare providers do not prevent providers in real-time from prescribing patients drugs that are unsafe for them, given their concurrent diseases and/or patient information. Thus, patients may be prescribed drugs that increase risks in patients as a result of drug-disease state interactions, and patients may subsequently experience associated side effects that are, at many times, unbeknownst to the patients or the healthcare providers. Additionally, EMR systems might not include drug-disease interaction information on every drug available for ordering, including updated information on new drugs and how new drugs may interact with various disease states in patients.

### BRIEF SUMMARY

Accordingly, there is a need for providing new systems and methods for detecting drug-disease interactions for patients to help improve patient safety. The present disclosure provides systems and methods that personalize medicine by screening for and alerting healthcare providers of moderate, major, and contraindicated drug-disease state interactions for patients, as well as generating recommendations with safer alternatives that healthcare providers can choose to minimize risks and/or harm to patients.

In an embodiment, an example method is described. The method includes receiving, at a computing platform and from a provider device of a provider, a first data request comprising data regarding a treatment requested for a patient based on a user input by the provider, in which the computing platform, the provider device, an electronic medical records (EMR) system, and a plurality of databases are communicatively coupled via a network. The method further includes determining, by at least one processor of the computing platform, a presence or absence of one or more contraindications or relevant precautionary conditions for the treatment by parsing at least one database in the plurality of databases based on the treatment, and upon determining the presence or absence of one or more contraindications or relevant precautionary conditions, determining values of a plurality of factors corresponding to the patient based on a real-time analysis of data obtained from the EMR system and one or more databases in the plurality of databases, in which the plurality of factors comprise at least one of an age, a gender, or a race of the patient, one or more concurrent drugs and/or herbal medicines used by the patient, one or more genetic variants of the patient, an adverse drug reaction history of the patient, pertinent lab and vitals data of the patient, and one or more disease states of the patient. The method further includes generating a total score by computing a total sum of at least a subset of the values of the plurality of factors corresponding to the patient, determining whether the total score is less than or greater than or equal to a predetermined threshold, and generating a recommendation for prescribing the treatment requested for the patient in response to the determination of the total score and an indication being treated in the patient. In response to determining that the total score is less than the predetermined threshold, a recommendation indicating a moderate drug-disease interaction for prescribing the treatment requested for the patient is generated. In response to determining that the total score is greater than or equal to the predetermined threshold, a recommendation indicating a major drug-disease interaction for prescribing the treatment requested for the patient is generated. The method further includes transmitting, to the provider device, a second data request for data regarding the indication being treated in the patient in response to the recommendation, receiving, from the provider device, the data regarding the indication being treated in the patient, determining, by the at least one processor, an availability or unavailability of one or more alternative treatments to replace the treatment requested for the patient for treating the indication in the patient, generating an additional recommendation for the patient based on the determination of the availability or the unavailability of the one or more alternative treatments, and receiving, from the provider device, an order for a selected treatment for the patient based on the additional recommendation, in which the order is transmitted to the EMR system.

In another embodiment, a drug-disease interaction detection server is described. The drug-disease interaction detection server includes a plurality of databases and at least one processor in which the drug-disease interaction detection server is communicatively coupled to a provider device and an electronic medical records (EMR) system via a network. The at least one processor is configured to receive, from the provider device of a provider, a first data request comprising data regarding a treatment requested for a patient based on a user input by the provider, determine a presence or absence of one or more contraindications or relevant precautionary conditions for the treatment by parsing at least one database in the plurality of databases based on the treatment, and upon determining the presence or absence of one or more contraindications or relevant precautionary conditions, determine values of a plurality of factors corresponding to the patient based on a real-time analysis of data obtained from the EMR system and one or more databases in the plurality of databases, in which the plurality of factors comprise at least one of an age, a gender, or a race of the patient, one or more concurrent drugs and/or herbal medicines used by the patient, one or more genetic variants of the patient, an adverse drug reaction history of the patient, pertinent lab and vitals data of the patient, and one or more disease states of the patient. The at least one processor is further configured to generate a total score by computing a total sum of at least a subset of the values of the plurality of factors corresponding to the patient, determine whether the total score is less than or greater than or equal to a predetermined threshold, and generate a recommendation for prescribing the treatment requested for the patient in response to the determination of the total score and an indication being treated in the patient. In response to determining that the total score is less than the predetermined threshold, a recommendation indicating a moderate drug-disease interaction for prescribing the treatment requested for the patient is generated. In response to determining that the total score is greater than or equal to the predetermined threshold, a recommendation indicating a major drug-disease interaction for prescribing the treatment requested for the patient is generated. The at least one processor is further configured to transmit, to the provider device, a second data request for data regarding the indication being treated in the patient in response to the recommendation, receive, from the provider device, the data regarding the indication being treated in the patient, determine an availability or unavailability of one or more alternative treatments to replace the treatment requested for the patient for treating the indication in the patient, generate an additional recommendation for the patient based on the determination of the availability or the unavailability of the one or more alternative treatments, and receive, from the provider device, an order for a selected treatment for the patient based on the additional recommendation, in which the order is transmitted to the EMR system.

In another embodiment, a provider device comprising a memory, a network interface, and at least one processor coupled to the memory is described. The provider device is communicatively coupled to a drug-disease interaction detection server and an electronic medical records (EMR) system via the network interface. The at least one processor is configured to receive a first user input comprising data regarding a treatment requested for a patient by a provider, encrypt the data regarding the treatment requested for the patient and patient data comprising a patient name and identification information of the patient using an encryption key, resulting in encrypted data, transmit the encrypted data to the drug-disease interaction detection server, and in response to transmitting the encrypted data, receive, from the drug-disease interaction detection server, a recommendation in real-time regarding the treatment requested for the patient, in which the recommendation provides information identifying one or more drug-disease interactions of the treatment requested for the patient.

Further features and advantages, as well as the structure and operation of various embodiments, are described in detail below with reference to the accompanying drawings. It is noted that the specific embodiments described herein are not intended to be limiting. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form a part of the specification, illustrate embodiments of the present disclosure and, together with the description, further serve to explain the principles of the disclosure and to enable a person skilled in the pertinent art to make and use the disclosure.

FIG. 1 illustrates an example diagram of a system for drug-disease interaction detection, according to embodiments of the present disclosure.

FIG. 2 illustrates an example diagram of a drug-disease interaction detection server, according to embodiments of the present disclosure.

FIG. 3 illustrates an example diagram of a provider device, according to embodiments of the present disclosure.

FIGS. 4A, 4B, and 4C illustrate an example flowchart diagram of a method for performing a real-time analysis for determining drug-disease interactions for a treatment of a patient, according to embodiments of the present disclosure.

FIG. 5 illustrates an example flowchart diagram of a method for determining drug-disease interactions using a drug-disease interaction detection server, according to embodiments of the present disclosure.

FIG. 6 illustrates an example flowchart diagram of a method for encrypting data and receiving recommendations regarding drug-disease interactions at a provider device, according to embodiments of the present disclosure.

FIG. 7 illustrates a block diagram of example components of a computer system, according to embodiments of the present disclosure.

Embodiments of the present disclosure will be described with reference to the accompanying drawings.

### DETAILED DESCRIPTION

Although specific configurations and arrangements are discussed, it should be understood that this is done for illustrative purposes only. A person skilled in the pertinent art will recognize that other configurations and arrangements can be used without departing from the spirit and scope of the present disclosure. It will be apparent to a person skilled in the pertinent art that this disclosure can also be employed in a variety of other applications.

It is noted that references in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of one skilled in the art to effect such feature, structure or characteristic in connection with other embodiments whether or not explicitly described.

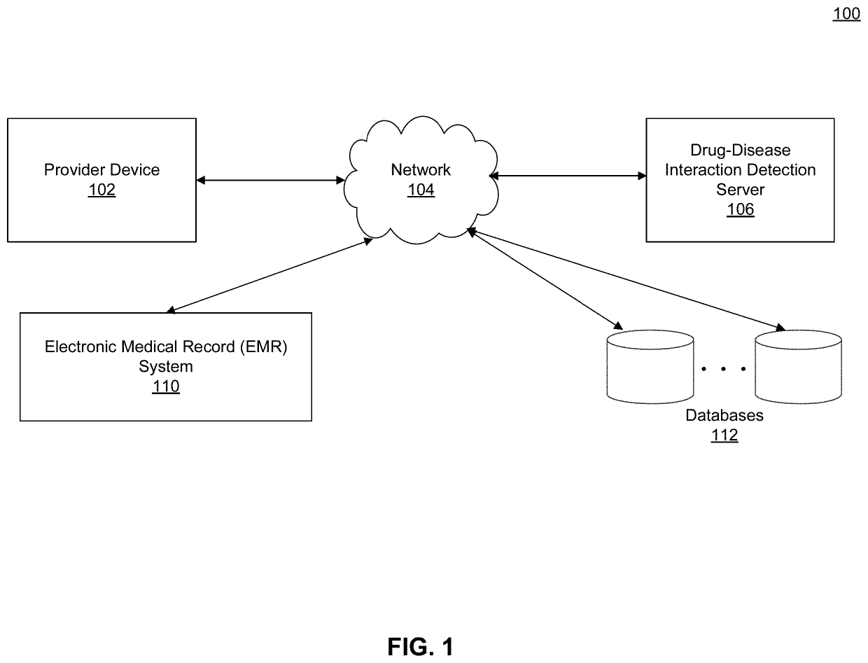

FIG. 1 illustrates an example diagram of a system 100 for drug-disease interaction detection, according to embodiments of the present disclosure. System 100 includes a provider device 102, a drug-disease interaction detection server 106, an electronic medical record (EMR) system 110, and a plurality of databases 112 communicatively coupled via a network 104.

Provider device 102 may be a personal digital assistant, desktop workstation, laptop or notebook computer, netbook, tablet, smart phone, mobile phone, smart watch or other wearable, or any combination thereof. Provider device 102 may be associated with a healthcare provider such as a physician, physician's assistant, nurse practitioner, clinical pharmacist, specialist, or the like. While only one provider device 102 is illustrated in FIG. 1 for reference, there may be any number of provider devices 102 in system 100, in which each provider device 102 is associated with a provider.

In some embodiments, provider device 102 may include one or more user interface devices, such as a keyboard, a mouse, a trackball, a touch pad, a touch screen, a pen or the like, video or touch free user interfaces, for interacting with a graphical user interface (GUI) provided on a display (e.g., a monitor screen, a liquid crystal display (LCD), a head-up display, a head-mounted display, etc.) in conjunction with information provided by drug-disease interaction detection server 106, EMR system 110, and/or the plurality of databases 112.

In some embodiments, provider device 102 may communicate with drug-disease interaction detection server 106, EMR system 110, and/or the plurality of databases 112 via network 104. Network 104 may be any one or any combination of a LAN (local area network), WAN (wide area network), telephone network, wireless network, point-to-point network, star network, token ring network, hub network, or other appropriate configuration. The network may comply with one or more network protocols, including an Institute of Electrical and Electronics Engineers (IEEE) protocol, a 3rd Generation Partnership Project (3GPP) protocol, a 4^{th} generation wireless protocol (4G) (e.g., the Long Term Evolution (LTE) standard, LTE Advanced, LTE Advanced Pro), a fifth generation wireless protocol (5G), and/or similar wired and/or wireless protocols, and may include one or more intermediary devices for routing data between provider device 102, drug-disease interaction detection server 106, and EMR system 110.

In some embodiments, drug-disease interaction detection server 106 may be integrated within EMR system 110 or may be a server that is separate from EMR system 110. Drug-disease interaction detection server 106 may be configured to communicate with and receive data requests from provider device 102 requesting treatments for patients, parse one or more databases in the plurality of databases 112 to determine one or more contraindications or relevant precautionary conditions for requested treatments, perform real-time analysis of data obtained from the EMR system 110 and/or plurality of databases 112 for identifying drug-disease interactions, as will be described in further detail below. As described herein, a “treatment” for a patient refers to any type of intervention or process performed on, or the administration of an active agent to, the patient with the objective of reversing, alleviating, ameliorating, inhibiting, slowing down or preventing the onset, progression, development, severity or recurrence of a symptom, complication or condition, or biochemical indicia associated with a disease. In some embodiments, a treatment may include a medication, a vaccine, a drug, and/or an herbal medicine. In some embodiments, the term “complementary medicines” may be used to refer to herbal medicines and/or vitamins. In other embodiments, herbal medicines may also include vitamins. As described herein, “contraindications” refer to conditions under which a particular drug or herbal product should not be administered as the risk of serious patient harm outweighs the benefit. As described herein, “relevant precautionary conditions” refer to conditions under which use of a particular drug or herbal product should be used with caution as its use may increase the risk of patient harm.

EMR system 110 may include any number of servers, computers, and/or devices that are configured to electronically store patient healthcare information. In some embodiments, EMR system 110 may aggregate data from various healthcare services and providers, such as hospitals, clinical care facilities, laboratories, radiology providers, and pharmacies. While only one EMR system 110 is illustrated in FIG. 1 for reference, there may be any number of EMR systems 110, in which each EMR system 110 is associated with one or more hospitals or other healthcare service center.

In some embodiments, EMR system 110 may comprise one or more EMR databases (not shown) that store patient data and medical history data regarding the health and treatment of patients. In some embodiments, one or more EMR databases in the EMR system 110 may store records for each patient, including demographics, medical history, medication and allergies, immunization status, laboratory test results, radiology images, vital signs, personal statistics like age and weight, and billing information for each patient. A provider may be able to access the stored data in the EMR system 110 using the provider device 102. Records in the EMR system 110 may include records of observational data, patient encounters, lab results, prescriptions, messages (e.g., messages transmitted to patients from their providers), and also biographical information about a patient, such as name, address, date of birth, and the like.

In some embodiments, one or more of the plurality of databases 112 in system 100 may be integrated within EMR system 110. For example, the plurality of databases 112 may include one or more EMR databases, which may be accessed by provider device 102 and/or drug-disease interaction detection server 106 for obtaining patient information. In other embodiments, the plurality of databases 112 may be separate from the EMR system 110. In some embodiments, the plurality of databases 112 may represent any number of databases, and may include a drug-disease interaction database.

In some embodiments, the drug-disease interaction database may comprise data regarding drug-disease interactions for various drugs and diseases. In some embodiments, the data regarding drug-disease interactions stored in the drug-disease interaction database may be based on established medical guidelines for treatment of patients with several medical conditions. For example, data from the drug-disease interaction database may indicate that nonsteroidal anti-inflammatory drugs (NSAIDs) should not be prescribed to patients with a history of chronic kidney disease because of increased drug concentrations from altered drug excretion, and therefore an increased susceptibility to adverse drug effects in this patient population (e.g., worsening glomerular filtration rate secondary to NSAIDs' interference with the autoregulation of renal blood flow). The drug-disease interaction database may include data regarding specific interactions between disease states and certain drugs that have been recognized within the medical community. In some embodiments, data in the drug-disease interaction database may be updated by providers (e.g., using provider device 102) as they encounter various medical cases and patients with varying underlying conditions. In other embodiments, data in the drug-disease interaction database may be updated based on receiving updates from an intelligent server (not pictured) configured to canvas, analyze, and determine various drug-disease interactions based on, for example, statistical or historical data in conjunction with machine learning algorithms and modeling.

In some embodiments, the plurality of databases 112 may further include a drug manufacturer database. The drug manufacturer database may include data provided by drug manufacturers for a plurality of drugs, including drug label information such as drug characteristics, dosage, interaction, cross-allergy information, and the like. In some embodiments, the data stored in the drug manufacturer database may be referred to as “package insert” information, including but not limited to a description of the drug, clinical pharmacology of the drug, pharmacokinetic information, pharmacodynamics, results from clinical studies, indications and usage, contraindications, warnings, precautions, drug interaction information, information on carcinogenesis, mutagenesis, and fertility, adverse reaction information, overdose information, dosage and administration information, and supply information (how the drug is supplied and/or delivered).

In some embodiments, the plurality of databases 112 may also include a medical literature database. The medical literature database may include data obtained from scientific articles and research publications from researchers, doctors, experts, and the like, regarding new drugs and treatments for specific diseases. The data stored in the medical literature database may include information on adverse reactions from new drugs and treatments in patients with various conditions and information on drug-disease interactions obtained from new clinical trials or studies.

In some embodiments, the data in the drug-disease interaction database, the drug manufacturer database, and/or the medical literature database may be compiled by drug-disease interaction detection server 106 and updated using one or more machine learning algorithms as described herein. In some embodiments, drug-disease interaction detection server 106 may generate a catalog of a plurality of medications comprising information regarding at least one of kinetic, dynamic, or pharmacogenomic interactions for each medication based on drug manufacturing information, medical literature, and established medical guidelines. Drug-disease interaction detection server 106 may update one or more of the databases in the plurality of databases 112 based on the catalog of the plurality of medications.

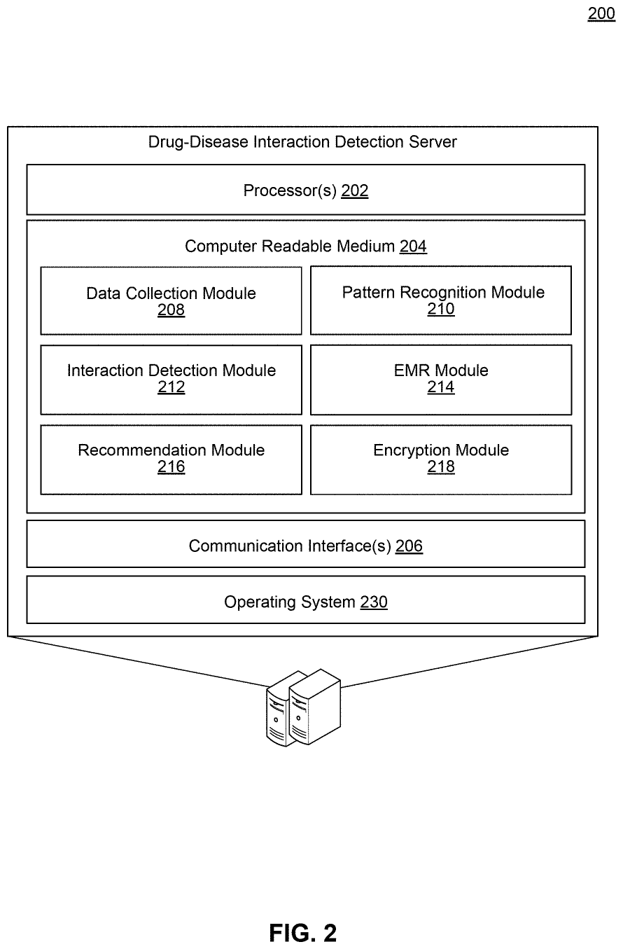

FIG. 2 illustrates a block diagram of drug-disease interaction detection server 200, according to embodiments of the present disclosure. Drug-disease interaction detection server 200 represents an exemplary embodiment of drug-disease interaction detection server 106 in FIG. 1. Drug-disease interaction detection server 200 includes one or more servers or other types of computing devices that can be embodied in any number of ways. For instance, the modules, other functional components, and data can be implemented on a single server, a cluster of servers, a server farm or data center, a cloud-hosted computing service, and so forth, although other computer architectures can additionally or alternatively be used.

Further, while the figures illustrate the components and data of the drug-disease interaction detection server 200 as being present in a single location, these components and data may alternatively be distributed across different computing devices and different locations in any manner. Consequently, the functions may be implemented by one or more computing devices, with the various functionality described above distributed in various ways across the different computing devices. Multiple drug-disease interaction detection servers 200 may be located together or separately, and organized, for example, as virtual servers, server banks and/or server farms. The described functionality may be provided by the servers of a single entity or enterprise, or may be provided by the servers and/or services of multiple different entities or enterprises.

In the illustrated example, the drug-disease interaction detection server 200 includes one or more processors 202, one or more computer-readable media 204, and one or more communication interfaces 206. Each processor 202 is a single processing unit or a number of processing units, and may include single or multiple computing units or multiple processing cores. The processor(s) 202 can be implemented as one or more microprocessors, microcomputers, microcontrollers, digital signal processors, central processing units, state machines, logic circuitries, and/or any devices that manipulate signals based on operational instructions. For instance, the processor(s) 202 may be one or more hardware processors and/or logic circuits of any suitable type specifically programmed or configured to execute the algorithms and processes described herein. The processor(s) 202 can be configured to fetch and execute computer-readable instructions stored in the computer-readable media 204, which can program the processor(s) 202 to perform the functions described herein.

The computer-readable media 204 include volatile and nonvolatile memory and/or removable and non-removable media implemented in any type of technology for storage of information, such as computer-readable instructions, data structures, program modules, or other data. Such computer-readable media 204 include, but are not limited to, RAM, ROM, EEPROM, flash memory or other memory technology, optical storage, solid state storage, magnetic tape, magnetic disk storage, network attached storage, storage area networks, cloud storage, or any other medium that can be used to store the desired information and that can be accessed by a computing device. Depending on the configuration of the drug-disease interaction detection server 200, the computer-readable media 204 may be a type of computer-readable storage media and/or may be a tangible non-transitory media to the extent that when mentioned, non-transitory computer-readable media exclude media such as energy, carrier signals, electromagnetic waves, and signals per se.

The computer-readable media 204 is used to store any number of functional components that are executable by the processors 202. In many implementations, these functional components comprise instructions or programs that are executable by the processors and that, when executed, specifically configure the one or more processors 202 to perform the actions attributed above to the drug-disease interaction detection server 200. In addition, the computer-readable media 204 store data used for performing the operations described herein.

In the illustrated example, the computer-readable media 204 further includes data collection module 208, pattern recognition module 210, interaction detection module 212, EMR module 214, recommendation module 216, and encryption module 218.

Data collection module 208 may collect data from different sources to compile in the plurality of databases 112 in system 100. In some embodiments, data collection module 208 may access and collect data from various sources, such as web sites and/or databases of drug manufacturers, regulatory authorities, government agencies (e.g., Food and Drug Administration (FDA)), federal regulations, science, research or medical journals, pharmacology textbooks or publications, and the like. In some embodiments, data collection module 208 may collect drug data regarding different drugs, vaccines, herbal medicines, or other treatments, as well as indication data corresponding to the drugs (e.g., data regarding different diseases or conditions that the drugs may be used to treat). In some embodiments, an indication for a drug may refer to the use of a specific drug for treating a particular disease. In some embodiments, the drug data and indication data collected by data collection module 208 may include text, graphs, figures, and the like obtained from various articles, publications, and guidelines from the different sources. In some embodiments, the data collected by data collection module 208 may be compiled and stored in the plurality of databases 112 and/or transmitted to pattern recognition module 210. In some embodiments, data collection module 208 may perform a pre-processing of the collected data before storing in the plurality of databases 112 and/or before transmitting to pattern recognition module 210. In some embodiments, the preprocessing step may include text mining, normalization, or other data analytics/processing steps performed of the collected data by the data collection module 208.

Pattern recognition module 210 may receive collected data from data collection module 208, perform classification, and identify one or more patterns in the data by applying a pattern recognition algorithm. In some embodiments, pattern recognition module 210 may be referred to herein as a classification module. In some embodiments, upon receiving the collected data from data collection module 208, pattern recognition module 210 may perform an extraction process to extract text blocks and/or keywords from parsing the collected data from scientific articles and websites and derive metadata. In some embodiments, pattern recognition module 210 may use the extracted text blocks and/or keywords and derived metadata along with the collected data to identify drug information for specific indications.

In some embodiments, the identified drug information obtained from the extraction process may comprise at least one of a description of the drug, clinical pharmacology of the drug, pharmacokinetic information, pharmacodynamics, results from clinical studies, indications and usage, contraindications, warnings, precautions, drug interaction information, information on carcinogenesis, mutagenesis, and fertility, adverse reaction information, overdose information, dosage and administration information, and the like. In some embodiments, pattern recognition module 210 may identify drug information from the collected data using keywords such as “risk,” “complication,” “contraindication,” “side effects,” “adverse reaction,” and the like. In some embodiments, pattern recognition module 210 may perform data processing and extraction processes by any combination of machine learning algorithms including but not limited to probabilistic models, neural networks, classifiers, frequency estimation, hidden Markov models, Gaussian mixture models, neural networks, matrix representation, Vector Quantization, or decision trees.

In some embodiments, pattern recognition module 210 may also train a pattern recognition algorithm, such as a neural network or classifier, to recognize patterns and provide updates for one or more databases in the plurality of databases 112 (e.g., drug-disease interaction database, drug manufacturer databases, and/or medical literature database). In some embodiments, pattern recognition module 210 may recognize which new drugs and disease states are most frequently associated with certain adverse drug reactions as reported in medical journals by identifying patterns in the collected data from data collection module 208. In some embodiments, pattern recognition module 210 may transmit updates to the plurality of databases 112 based on the identified patterns, such that one or more databases in the plurality of databases 112 are dynamically updated to include new information on drug-disease interactions and contraindications as clinicians and researchers come across new cases and report them. In some embodiments, pattern recognition module 210 may dynamically update one or more databases in the plurality of databases 112 based on the identified patterns in real-time, every week, every other week, every month, or another predetermined period of time.

Interaction detection module 212 may perform real-time analysis and drug-disease interaction detection for a treatment requested for a patient by provider device 102. In some embodiments, interaction detection module 212 may receive a request for data regarding treatment requested for a patient from provider device 102. In some embodiments, the data request may include the name of a requested treatment and information regarding the indication associated with the requested treatment. In some embodiments, the data request may also include patient data including a patient name and identification information, such as an identification number, a medical record number, a date of birth, or a phone number of the patient.

Upon receiving a request for a treatment for a patient, the interaction detection module 212 may perform a determination of whether one or more contraindications or relevant precautionary conditions are present or absent for the requested treatment. In some cases, certain drug-disease states may be contraindicated, such that certain drugs should not be prescribed to patients with particular conditions or diseases. Interaction detection module 212 may identify such contraindications by parsing data in one or more databases in the plurality of databases 112. In some embodiments, interaction detection module 212 may query a drug-disease interaction database in the plurality of databases 112 to identify any known contraindications or precautionary conditions that may exist for the treatment.

For example, the provider device 102 may transmit a data request for a medication for treating diabetes in a patient. Interaction detection module 212 may query the drug-disease interaction database in the plurality of databases 112 to determine that the medication is contraindicated in patients with kidney disease (e.g., stage 3b) and that patients with kidney disease should not be prescribed the medication. In another example, interaction detection module 212 may query the drug-disease interaction database to identify that a medication requested by the provider device 102 to treat a first disease counteracts and/or alters the disease state of a second disease in the patient. In some embodiments, interaction detection module 212 may communicate with recommendation module 216 to generate one or more recommendations for the provider device 102 regarding the drug-disease interactions for a particular treatment and whether or not the particular treatment should be prescribed to the patient, as will be described in further detail below.

In addition to querying databases for drug-disease information, interaction detection module 212 may also perform a scoring of a plurality of factors that are relevant for determining contraindications or precautionary conditions for a requested treatment for a patient. In some embodiments, the plurality of factors may include at least one of an age, a gender, or a race of a patient, one or more concurrent drugs and/or herbal medicines used by the patient, one or more genetic variants of the patient, an adverse drug reaction history of the patient, pertinent lab data of the patient, and one or more disease states of the patient. In some embodiments, the patient's vitals, such as blood pressure, heart rate, respiratory rate, weight, body mass index (BMI), and the like, may be included in the plurality of factors. In some embodiments, pertinent lab data of the patient may include data regarding complete blood count (CBC), prothrombin time, basic metabolic panel, comprehensive metabolic panel, lipid panel, liver function test, thyroid panels, hemoglobin A1C, urinalysis, and cultures obtained from the patient. In some embodiments, factors may be weighted in relation to other factors. For example, certain demographic factors may be given more weight based on the system identifying a high risk associated with said demographics and other factors, such as blood pressure, BMI, or the like.

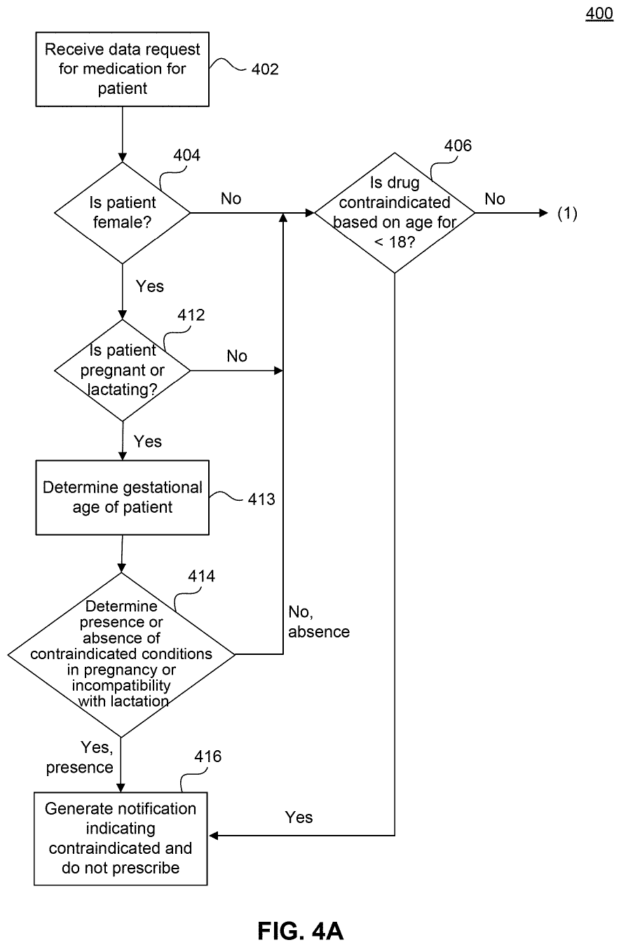

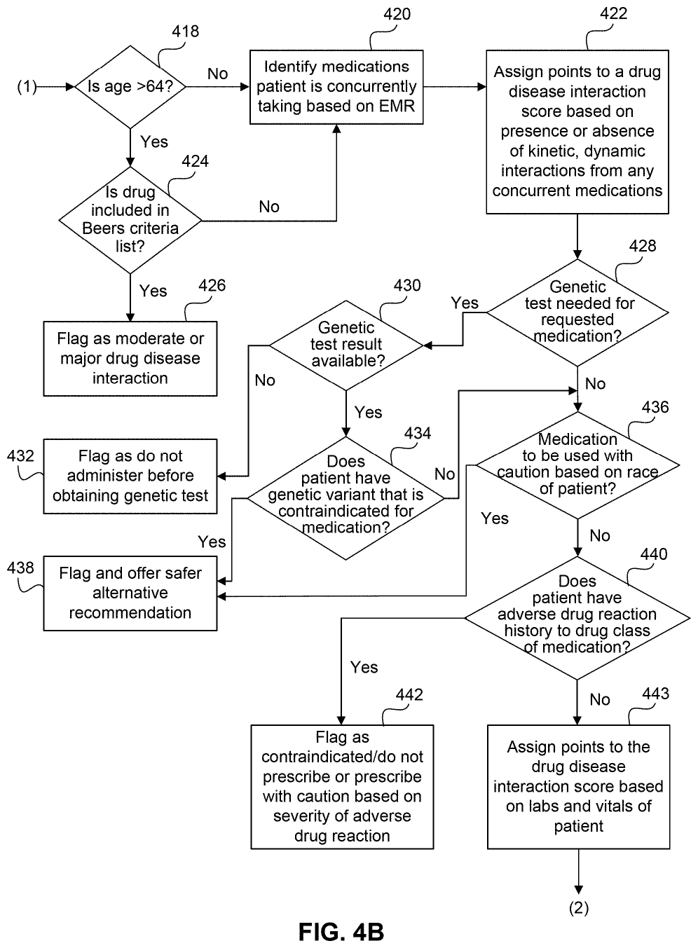

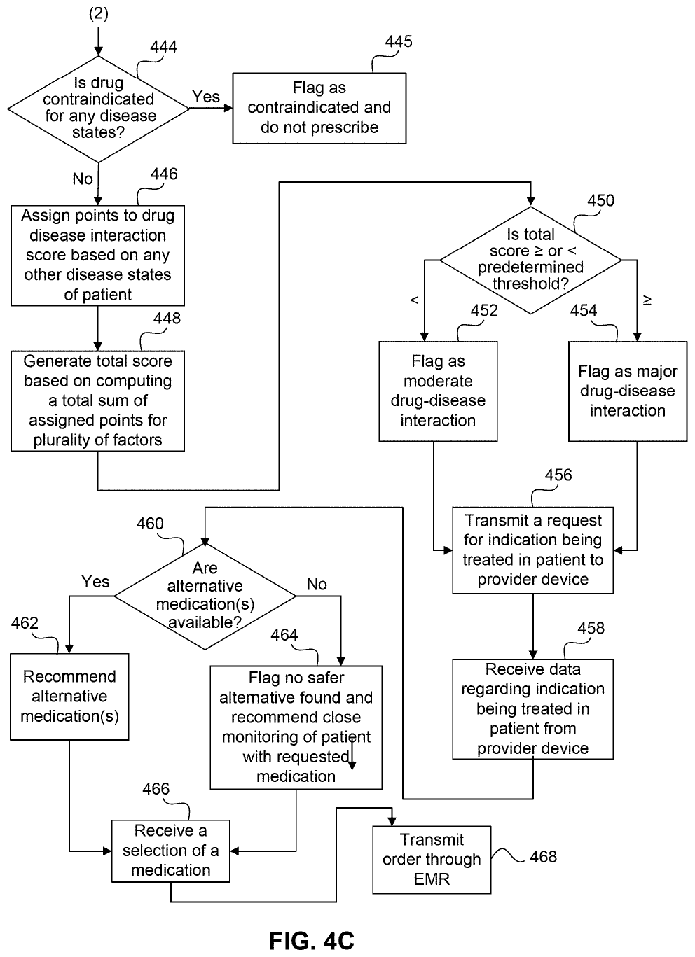

In some embodiments, interaction detection module 212 may calculate a total score for the drug-disease interactions for a requested treatment for a patient, in which the total score may be calculated as a sum of the values of the plurality of factors. In some embodiments, the total score may be calculated based on a subset of values of the plurality of factors. In some embodiments, the total score may be calculated as a weighted combination of the factor values. In some embodiments, each factor may be associated with a different weight (or a ranking) that is used to rank or prioritize the level of importance that each factor may have in the calculation of drug-disease interaction detection scores. For example, a first factor of a patient's age may have a higher weight than a second factor of a patient's glucose value. In some embodiments, interaction detection module 212 may output scores for each of a plurality of factors and produce an aggregate output score based on the output scores. In some embodiments, interaction detection module 212 may use an algorithm for performing the scoring with one or more weighting factors based on user input from the provider device 102, as described in further detail with respect to FIGS. 4A-4C below.

In some embodiments, interaction detection module 212 may perform the calculations and drug-disease interaction detection based on extracting data from one or more databases in the plurality of databases 112 and/or EMR system 110. In some embodiments, interactions detection module 212 may communicate with EMR module 214 (which interfaces with EMR system 110) to access patient information from the patient's electronic medical record and identify values corresponding to factors used in the real-time data analysis.

EMR module 214 may communicate and interface with EMR system 110 in providing the drug-disease interaction detection server 200 and/or provider device 102 with access to patient data and medical history data. In some embodiments, EMR module 214 may access patient data from electronic health records in EMR system 110 for medical records of patients as requested by the provider device 102 and further communicate with encryption module 218 to encrypt patient data before transmitting to provider device 102. In some embodiments, patient data that is received from EMR system 110 at EMR module 214 may be encrypted. Encryption module 218 may be configured to perform decryption of the patient data.

Encryption module 218 may perform encryption and decryption of data transmitted between drug-detection interaction detection server 106, provider device 102, EMR system 110, and the plurality of databases 112. In some embodiments, encryption module 218 may perform encryption and decryption of data using one or more encryption keys and/or cryptographic algorithms. By performing encryption and decryption of data, the encryption module 218 may allow for protection of patient information and communications between drug-disease interaction and detection server 200, EMR system 110, provider device 102, and/or databases 112, in accordance with the Health Insurance Portability and Accountability Act (HIPAA).

Recommendation module 216 may generate alerts, notifications, and/or recommendations for healthcare providers based on the drug-disease interaction detection. In some embodiments, recommendation module 216 may generate and transmit one or more recommendations to provider device 102. In some embodiments, recommendation module 216 may receive data regarding a total score for the drug-disease interactions for a requested treatment for a patient from interaction detection module 212 and generate a recommendation based on the total score determined and an indication (e.g., a condition or disease) being treated in the patient. In some embodiments, if the total score is less than or equal to a predetermined threshold, recommendation module 216 generates a recommendation indicating a moderate drug-disease interaction for prescribing the treatment requested for the patient. In some embodiments, if the total score is greater than the predetermined threshold, recommendation module 216 generates a recommendation indicating a major drug-disease interaction for prescribing the treatment requested for the patient. In some embodiments, recommendation module 216 generates recommendations to provider device 102 based on identifying one or more contraindications of a requested treatment for a patient.

In addition to providing alerts or recommendations regarding drug-disease interactions for a requested treatment, recommendation module 216 may also access one or more databases in plurality of databases 112 and identify one or more alternative treatments to replace the requested treatment for a patient. In some embodiments, there may be one or more alternative drugs or medications that are safer to prescribe the patient than the requested treatment. For example, one or more alternative drugs or medications may have less drug-disease interactions than a requested treatment that interacts with a patient's preexisting disease states. Thus, recommendation module 216 may determine whether or not any alternative drugs or medications are available, generate recommendations that are personalized according to the patient's information, and transmit recommendations to provider device 102.

In some embodiments, recommendation module 216 may transmit various alerts, notifications, and/or recommendations to provider device 102, including but not limited to: an alert indicating “contraindicated” and “do not prescribe,” an alert indicating “major drug-disease state interaction,” an alert indicating “moderate drug-disease state interaction,” an alert indicating “do not administer before obtaining genetic testing,” an alert indicating “prescribe with caution,” an alert indicating “no safer alternative found” and “close monitoring recommended,” an alert indicating “drug level out of range,” and the like. In addition to providing the alerts, recommendation module 216 may include a description of the drug-disease interactions and corresponding issues found, along with information on one or more alternative treatments that may be safer for the patient. In some embodiments, recommendation module 216 may rank the one or more alternative treatments found, based on how safe each alternative treatment is for the patient, and include the ranking in the recommendation transmitted to provider device 102.

Additional functional components stored in the computer-readable media 204 include an operating system 230 for controlling and managing various functions of the drug-disease interaction detection server 200. The drug-disease interaction detection server 200 also includes or maintains other functional components and data, such as other modules and data, which include programs, drivers, and the like, and the data used or generated by the functional components. Further, the drug-disease interaction detection server 200 includes many other logical, programmatic and physical components, of which those described above are merely examples that are related to the discussion herein.

The communication interface(s) 206 include one or more interfaces and hardware components for enabling communication with various other devices, including provider device 102, EMR system 110, databases 112, or other computing devices over network 104. For example, communication interface(s) 206 facilitate communication through one or more of the Internet, cable networks, cellular networks, wireless networks (e.g., Wi-Fi, cellular) and wired networks. As several examples, the drug-disease interaction detection server 200 and other devices communicate and interact with one another using any combination of suitable communication and networking protocols, such as Internet protocol (IP), transmission control protocol (TCP), hypertext transfer protocol (HTTP), cellular or radio communication protocols, and so forth. Examples of communication interface(s) include a modem, a network interface (such as an Ethernet card), a communications port, a Personal Computer Memory Card International Association (PCMCIA) slot and card, and the like.

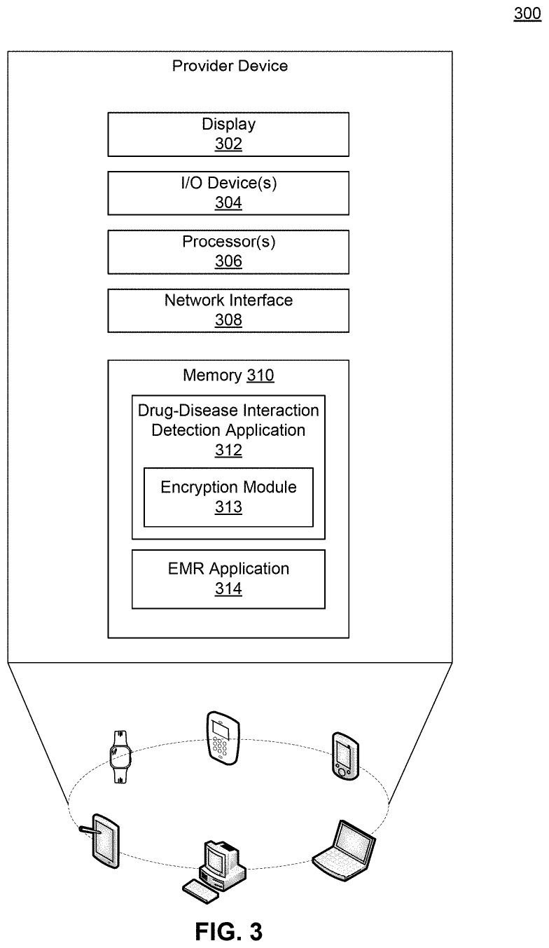

FIG. 3 illustrates an example diagram of a provider device 300, according to embodiments of the present disclosure. Provider device 300 represents an exemplary embodiment of provider device 102 in FIG. 1. Provider device 300 includes display 302, input/output (I/O) devices 304, one or more processors 306, network interface 308, and memory 310.

Display 302 of provider device 300 may employ any suitable display technology depending on the type of device used as the provider device 300. For example, the display 302 may be a liquid crystal display, a light emitting diode display, or any other suitable type of display able to present digital content thereon. In some embodiments, the display 302 includes a touch sensor with the display to provide a touchscreen display configured to receive touch inputs for enabling interaction with a graphical user interface presented on the display 302. Accordingly, implementations herein are not limited to any particular display technology.

Provider device 300 may be equipped with various input/output (I/O) devices 304. Such I/O devices 304 include various user interface controls, such as buttons, joystick, keyboard, mouse, touch screen, and the like, audio speakers, connection ports, and so forth. In some embodiments, a provider may use I/O devices 304 to provide one or more user inputs related to requesting drug-disease interaction data regarding a particular treatment for a patient. In some embodiments, a provider may use I/O devices 304 to submit data regarding a requested treatment for a patient and/or patient data such as a patient name and identification information to drug-disease interaction detection server 200, as well as an order for a selected treatment for a patient to EMR system 110. Additionally, provider device 300 may include various other components that are not shown, examples of which include removable storage, a power source, such as a battery and power control unit, and so forth.

Provider device 300 also includes or maintains other functional components and data, such as other modules and data, which include programs, drivers, and the like, and the data used or generated by the functional components. Further, provider device 300 includes many other logical, programmatic and physical components, of which those described above are merely examples that are related to the discussion herein.

Network interface 308 includes one or more interfaces and hardware components for enabling communication with various other devices, such as EMR system 110, drug-disease interaction detection server 106 or 200 and the plurality of databases 112, over network 104. For example, network interface 308 facilitates communication through one or more of the Internet, cable networks, cellular networks, wireless networks (e.g., Wi-Fi, cellular) and wired networks. In some embodiments, drug-disease interaction detection server 200 and provider device 300 communicate and interact with one another using any combination of suitable communication and networking protocols, such as Internet protocol (IP), transmission control protocol (TCP), hypertext transfer protocol (HTTP), cellular or radio communication protocols, and so forth. Examples of communication interface(s) include a modem, a network interface (such as an Ethernet card), a communications port, a PCMCIA slot and card, and the like.

Provider device 300 further includes one or more processors 306. Each processor 306 is a single processing unit or a number of processing units, and may include single or multiple computing units or multiple processing cores. The processor(s) 306 can be implemented as one or more microprocessors, microcomputers, microcontrollers, digital signal processors, central processing units, state machines, logic circuitries, and/or any devices that manipulate signals based on operational instructions. For instance, the processor(s) 306 may be one or more hardware processors and/or logic circuits of any suitable type specifically programmed or configured to execute the algorithms and processes described herein. The processor(s) 306 can be configured to fetch and execute computer-readable instructions stored in the memory 310, which can program the processor(s) 306 to perform the functions described herein.

In some embodiments, memory 310 may represent a computer-readable media that may include volatile and nonvolatile memory and/or removable and non-removable media implemented in any type of technology for storage of information, such as computer-readable instructions, data structures, program modules, or other data. Such computer-readable media include, but are not limited to, RAM, ROM, EEPROM, flash memory or other memory technology, optical storage, solid state storage, magnetic tape, magnetic disk storage, network attached storage, storage area networks, cloud storage, or any other medium that can be used to store the desired information and that can be accessed by a computing device. Depending on the configuration of the provider device 300, the computer-readable media may be a type of computer-readable storage media and/or may be a tangible non-transitory media to the extent that when mentioned, non-transitory computer-readable media exclude media such as energy, carrier signals, electromagnetic waves, and signals per se.

The computer-readable media may be used to store any number of functional components that are executable by the processors 306. In many implementations, these functional components comprise instructions or programs that are executable by the processors and that, when executed, specifically configure the one or more processors 306 to perform the actions described herein below. In addition, the computer-readable media may store data used for performing the operations described herein below.

Memory 310 further includes drug-disease interaction detection application 312 and EMR application 314. Drug-disease interaction detection application 312 is an application that is configured to interface with drug-disease interaction detection server 200. In some embodiments, drug-disease interaction detection application 312 may transmit data to and receive interaction data from drug-disease interaction detection server 200. For example, a provider associated with provider device 300 may use the drug-disease interaction detection application 312 to access a user interface (e.g., shown on display 302) and submit a request for drug-disease interaction data for a treatment requested for a patient. In some embodiments, the request may include a name of the requested treatment and patient data including a patient name and identification information of the patient. In some embodiments, the identification information of the patient may include at least one of an identification number, a medical record number, a date of birth, or a phone number of the patient.

Drug-disease interaction detection application 312 further includes an encryption module 313 that is configured to encrypt and decrypt data that is transmitted between provider device 102, drug-disease interaction detection server 200, EMR system 110, and the plurality of databases 112 over network 104. In some embodiments, encryption module 313 may be configured to perform encryption and decryption of data using one or more encryption keys and/or cryptographic algorithms. In response to transmitting data requests with encrypted data, drug-disease interaction detection application 312 may receive recommendations in real-time regarding treatments requested for patients from the drug-disease interaction detection server 200. In some embodiments, the recommendation received at the drug-disease interaction detection application 312 may include information identifying one or more drug-disease interactions of treatments requested for the patients. Additionally, drug-disease interaction detection application 312 may also receive, from drug-disease interaction detection server, data regarding or more alternative treatments to prescribe a patient based on one or more drug-disease interactions of a treatment requested for the patient and based on an indication associated with the treatment requested for the patient.

EMR application 314 is an application that is configured to interface with EMR system 110 and/or EMR module 214 in the drug-disease interaction detection server 200. In some embodiments, EMR application 314 may allow the provider to access patient data and medical history data from electronic health records in EMR system 110. In some embodiments, the EMR application 314 may allow the provider to manage patient care, clinical charting, billing, and the like. In some embodiments, the provider may use the EMR application 314 to transmit an order for a prescription or a treatment for a particular patient, in which the order may be received by EMR system 110 and/or EMR module 214 in the drug-disease interaction detection server 200.

FIGS. 4A, 4B, and 4C illustrate an example flowchart diagram of a method 400 for performing a real-time analysis for determining drug-disease interactions for a treatment of a patient, according to embodiments of the present disclosure. Method 400 may be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIGS. 4A-4C, as will be understood by a person of ordinary skill in the art(s). For reference, steps 402-416, 418-443, and 444-468 are shown in FIGS. 4A, 4B, and 4C, respectively.

At step 402, a data request for a medication for a patient is received. In some embodiments, the data request for the patient may be transmitted from provider device 300 and received by drug-disease interaction detection server 200. At step 404, the drug-disease interaction detection server 200 may determine if the patient is female. If the patient is not female, then the method 400 in this example proceeds to step 406. If the patient is female, then the method 400 in this example proceeds to step 412. At step 412, the drug-disease interaction detection server 200 may determine if the patient is pregnant or lactating. If the patient is not pregnant or lactating, then the method 400 in this example proceeds to step 406. If the patient is pregnant or lactating, then the method 400 in this example proceeds to step 413.

At step 413, the drug-disease interaction detection server 200 may determine a gestational age of a pregnant patient. In some embodiments, certain drugs or medications may be contraindicated or prescribed with caution to patients based on the gestational age or trimester of a pregnancy in the patient. In some embodiments, the drug-disease interaction detection server 200 may determine whether a patient is in a first trimester (0-3 months), second trimester (4-6 months), or a third trimester (7-9 months) of a pregnancy. If the patient is lactating and not pregnant, the drug-disease interaction detection server 200 may omit step 413 and proceed from step 412 to step 414. At step 414, the drug-disease interaction detection server 200 may determine a presence or absence of any contraindicated conditions for the requested medication for pregnant or lactating women. In some embodiments, the drug-disease interaction detection server 200 may determine whether the requested medication is incompatible with lactation. In some embodiments, the drug-disease interaction detection server 200 may determine whether the requested medication is contraindicated for a patient based on the determined gestational age of the patient. If there are contraindicated conditions present for pregnant or lactating women, then the method 400 in this example proceeds to step 416. At step 416, the drug-disease interaction detection server 200 may generate a notification indicating that the medication is contraindicated and provide a recommendation for the provider not to prescribe the medication to the patient.

If there are no contraindicated conditions present for pregnant or lactating women, then the method 400 in this example proceeds to 406. At step 406, the drug-disease interaction detection server 200 may determine if the medication or drug is contraindicated based on an age of the patient being less than 18 years old. If the medication is contraindicated based on the patient's age being less than 18 years old, the method 400 in this example proceeds back to step 416, where the notification regarding the medication being contraindicated is generated along with the recommendation for the provider not to prescribe.

If the medication is not contraindicated based on the patient's age being less than 18 years old, then the method 400 in this example proceeds to step 418 as shown in FIG. 4B. At step 418, the drug-disease interaction detection server 200 may determine if the age of the patient is greater than 64 years old. If the patient's age is not greater than 64 (e.g., the patient is less than 65 years old), then the method 400 in this example proceeds to step 420. If the patient's age is greater than 64, then the method 400 in this example proceeds to step 424. At step 424, the drug-disease interaction detection server 200 may determine if the requested medication for the patient is included on the Beer's criteria list. In some embodiments, the drug-disease interaction detection server 200 may access medical guidelines data stored in one or more databases 112 to determine if a medication is contraindicated based on age. For example, the drug-disease interaction detection server 200 may access data regarding the Beer's criteria list comprising a list of medications that may be unsafe and/or medications that providers are advised to prescribe with caution for patients in a particular age group (e.g., patients 65 and older) because of the risk of adverse effects.

If the medication is not included in the Beer's criteria list, then the method 400 in this example proceeds to step 420. If the medication is included in the Beer's criteria list, then the method 400 in this example proceeds to step 426. At step 426, the drug-disease interaction detection server 200 may generate a recommendation in which the medication is flagged for having a moderate or major drug-disease interaction. In some embodiments, the recommendation may include a description of the drug-disease interaction found and may also provide one or more alternative treatments for the patient based on the patient's age. At step 420, the drug-disease interaction detection server 200 may identify any medications that the patient is concurrently taking based on accessing the EMR system 110. In some embodiments, the drug-disease interaction detection server 200 may determine if the patient is taking any concurrent drugs, medications, and/or herbal medicines.

At step 422, the drug-disease interaction detection server 200 may assign points to a drug-disease interaction score based on the presence or absence of kinetic and dynamic interactions from any concurrent medications. In some embodiments, the drug-disease interaction score may be based on scores corresponding to a plurality of factors, including scores corresponding to kinetic interactions, dynamic interactions, drug reaction history, kidney function, liver function, hemoglobin A1C, drug levels, and/or disease states, as described herein. In some embodiments, kinetic interactions may indicate that one medication may induce or inhibit the metabolism of another medication by increasing the liver's ability to metabolize. For example, various medications can enhance or inhibit production of certain drug-metabolizing enzymes in the liver. In some embodiments, the drug-disease interaction detection server 200 may identify different degrees of interactions, including major cytochrome P450(CYP) and/or minor CYP interactions. In some embodiments, major CYP inhibitors may be assigned higher points than moderate CYP inhibitors. For example, if a medication is determined to have major CYP inhibition or induction with another concurrent drug that the patient is taking, then the drug-disease interaction detection server 200 may assign 2 points to a kinetic interaction factor/score. If the medication is determined to have moderate CYP inhibition or induction with another concurrent drug that the patient is taking, then the drug-disease interaction detection server 200 may assign 1 point to a kinetic interaction factor/score. If the drug-disease interaction detection server 200 determines that the patient is taking multiple drugs that have a kinetic interaction, then the kinetic interaction/factor score may be multiplied by a factor corresponding to the number of drugs.

In some embodiments, the drug-disease interaction detection server 200 may identify dynamic interactions including pharmacologic interactions and pharmacodynamics (e.g., additive and/or antagonistic pharmacologic effects). In some embodiments, certain drugs may be members of different drug classes but may work on similar pathways or similar receptors. For example, a patient may already be prescribed a first drug that increases the levels of a neurotransmitter that suppresses nerve impulses, and the medication that is being requested for the patient may be a second drug that interacts with the first drug. The drug-disease interactions between the first and second drugs may result in an increased risk of central nervous system depression in the patient, resulting in an additive pharmacologic effect. In another example, a first and second drug prescribed for a patient may block or negate each other's effects in a patient, resulting in an antagonistic pharmacologic effect.

Thus, the drug-disease interaction detection server 200 may identify if an additive pharmacologic effect and/or antagonistic pharmacologic effect exists based on the concurrent medications of the patient. If an additive pharmacologic effect exists, the drug-disease interaction detection server 200 may assign 1 point to a dynamic factor/score. If an antagonistic pharmacologic effect exists, the drug-disease interaction detection server 200 may assign 1 point to a dynamic factor/score. If neither additive nor antagonistic pharmacologic effect exists, no points are assigned. If the drug-disease interaction detection server 200 determines that the patient is taking multiple drugs that have a dynamic interaction, then the dynamic interaction/factor score may be multiplied by a factor corresponding to the number of drugs. In some embodiments, the drug-disease interaction detection server 200 may determine kinetic and dynamic interactions based on accessing drug manufacturer data and package insert data stored in the plurality of databases 112 and identifying or comparing interactions corresponding to the requested treatment for the patient and the concurrent medications the patient is taking.

At step 428, the drug-disease interaction detection server 200 may determine whether a genetic or genomic test is needed before prescribing the requested medication for the patient. In some embodiments, providers may obtain genotypes of a patient to assess if the patient is more prone to having a drug-disease interaction based on the presence or absence of certain alleles in the patient's cells (e.g., genetic variants). In some embodiments, specific drugs may be used with caution or avoided for patients that have certain genetic variants because patients with the certain genetic variants may be more at risk for an allergic or adverse reaction to the specific drugs. For example, Abacavir (a medication used to treat HIV/AIDS) is contraindicated in patients who are HLA-B*5701positive, so testing/screening for the HLA-B*5701allele is recommended prior to ordering Abacavir for patients.

Thus, patients may need to be genetically tested for a genotype before being prescribed certain drugs. If a genetic or genomic test is not needed, then the method 400 in this example proceeds to step 436. If a genetic or genomic test is needed, then the method 400 in this example proceeds to step 430. At step 430, the drug-disease interaction detection server 200 may determine if a genetic test result is available. In some embodiments, the drug-disease interaction detection server 200 may access the EMR system 110 to determine if a genetic test result is available in the corresponding electronic medical record of the patient. If a genetic test result is unavailable, then the method 400 in this example proceeds to step 432. If a genetic test result is available, then the method 400 in this example proceeds to step 434.

At step 432, the drug-disease interaction detection server 200 may generate a recommendation in which the medication is flagged to not be administered to the patient before obtaining a genetic test. In some embodiments, the recommendation may include a description of the genetic test to be administered and a genetic variant that may be problematic for the requested medication (e.g., causing allergic or adverse drug reactions). At step 434, the drug-disease interaction detection server 200 may determine whether the patient has the genetic variant that is contraindicated for the requested medication based on the patient's genetic test result. For example, the drug-disease interaction detection server 200 may determine if the patient is positive for the HLA-B*5701allele before Abacavir can be prescribed. If the patient has the genetic variant, then the method 400 in this example proceeds to step 438.

At step 438, the drug-disease interaction detection server 200 may generate a recommendation in which the medication is flagged as contraindicated and the provider is advised via an alert not to prescribe the medication. If the patient does not have the genetic variant, then the method 400 in this example proceeds to step 436. At step 436, the drug-disease interaction detection server 200 may determine whether the medication should be used with caution based on the race and/or ethnicity of the patient. For example, African American patients may be more at risk for sudden death from certain drugs (e.g., Serevent) than compared to the general population. In other cases, Asian Americans and Pacific Islander patients may be more at risk for treatment failure to a certain drug (e.g., Plavix) because such patients might not have the particular CYP P450enzyme needed to convert the prodrug to the active moiety in their bodies.

In some embodiments, the drug-disease interaction detection server 200 may access the corresponding electronic medical record of the patient in the EMR system 110 to obtain race and/or ethnicity data of the patient (which may be self-identified by the patient during patient intake), as well as drug manufacturer data and package insert data stored in the plurality of databases 112 to identify if there are any precautions of how the requested medication may affect patients of certain races/ethnicities. If the drug-disease interaction detection server 200 determines that the medication should be used with caution based on identifying the race and/or ethnicity of the patient, then the method 400 in this example proceeds to step 438, where the medication is flagged and a notification generated to the provider offering to recommend a safer alternative.. If the drug-disease interaction detection server 200 determines that the medication does not have any precautions based on identifying the race and/or ethnicity of the patient, then the method 400 in this example proceeds to step 440.

At step 440, the drug-disease interaction detection server 200 may determine if the patient has a history of adverse drug reactions to the drug class of the requested medication. In some embodiments, the drug-disease interaction detection server 200 may access the corresponding electronic medical record of the patient in the EMR system 110 to identify if the patient has had any adverse drug reactions to other medications in the same drug class as the drug class of the requested medication. For example, a patient may have a history of angioedema with the use of Lisinopril and adverse drug reactions to any medications that are angiotensin converting enzyme inhibitors (ACE inhibitors), and the drug-disease interaction detection server 200 may detect this by retrieving this medical history data from the EMR system 110. In some embodiments, the drug-disease interaction detection server 200 may assign 1 point to an adverse drug reaction history factor/score if the drug-disease interaction detection server 200 identifies an adverse drug reaction history to the prescribed drug class of the requested medication. In some embodiments, identifying a history of adverse drug reactions to the drug class also includes identifying the patient's history of allergic reactions to any medications in the drug class.

If the patient has a history of adverse drug reactions to the drug class, then the method 400 in this example proceeds to step 442. At step 442, the drug-disease interaction detection server 200 may generate a recommendation in which the medication is flagged as contraindicated, and the provider may be advised not to prescribe the medication. For example, the drug-disease interaction detection server 200 may recommend that a provider not order or prescribe a medication that is an ACE inhibitor or an angiotensin II receptor blocker (ARB) for lowering blood pressure in a patient if the drug-disease interaction detection server 200 detects that the patient has a medical history of angioedema from another ACE inhibitor or ARB. In some embodiments, the recommendation may include a description of the patient's history of adverse drug reactions to the prescribed drug class, and the provider may be advised not to prescribe the medication or to prescribe the medication with caution based on the severity of the patient's history of adverse drug reactions. For example, the drug-disease interaction detection server 200 may provide a recommendation indicating a precautionary condition if a drug that is an ACE inhibitor is requested for a patient with a history of cough from another drug that is an ACE inhibitor. In such cases, the recommendation may flag the medication to be prescribed with caution by the provider. In some embodiments, the recommendation generated at step 442 may include a recommendation for an alternative drug class to prescribe the patient. In some embodiments, the drug-disease interaction detection server 200 may automatically generate one or more notifications or recommendations upon flagging a medication as contraindicated or flagging a medication as to be prescribed with caution. In some embodiments, the flagging of a medication by drug-disease interaction detection server 200 may activate an automatic transmission of a notification or recommendation to the provider device 300 based on the flagging.

If the patient does not have a history of adverse drug reactions to the drug class, then the method 400 in this example proceeds to step 443. At step 443, the drug-disease interaction detection server 200 assigns points to the drug-disease interaction score based on labs and vitals of the patients. In some embodiments, the drug-disease interaction detection server 200 may obtain lab data and vitals data of a patient, such as blood pressure, heart rate, respiratory rate, weight, body mass index (BMI), and the like, from the EMR system 110. In some embodiments, lab data of the patient may include data regarding kidney function, liver function, one or more hemoglobin levels, and other data obtained from labs of the patient. In some embodiments, lab data of the patient may further include data regarding complete blood count (CBC), prothrombin time, basic metabolic panel, comprehensive metabolic panel, lipid panel, liver function tests, thyroid panels, hemoglobin A1C, urinalysis, and/or cultures obtained from the patient.

In some embodiments, the drug-disease interaction detection server 200 may assess kidney function of the patient based on the glomerular filtration rate (GFR) levels, and the drug-disease interaction detection server 200 may assign a number of points to a kidney function factor/score according to the GFR levels. In some embodiments, the number of points assigned to the kidney function factor/score may increase with declining kidney function if the requested medication or drug is renally eliminated and necessitates a renal dose adjustment. In some embodiments, the drug-disease interaction detection server 200 may assign points for the kidney function factor/score according to the levels shown in Table 1 below. In some embodiments, creatinine clearance may also be included and calculated based on GFR.

TABLE 1
Kidney Function Levels and Corresponding Points Assigned

| GFR Level | Kidney Condition | Points Assigned |
|---|---|---|
| G1 >90 | Normal | 0 points |
| G2 60-89 | Mildly decreased | 1 point |
| G3a 45-59 | Mild-moderately decreased | 2 points |
| G3b 30-44 | Moderately-severely decreased | 3 points |
| G4 15-29 | Severely decreased | 4 points |
| G5 <15 | Kidney failure | 5 points |

In some embodiments, the drug-disease interaction detection server 200 may assess liver function of the patient by measuring protein and enzyme levels of the liver and assigning a number of points to a liver function factor/score according to the protein and enzyme levels. In some embodiments, the number of points assigned to the liver function factor/score may increase with declining liver function if the requested medication or drug is hepatically metabolized and necessitates a hepatic dose adjustment. In some embodiments, the drug-disease interaction detection server 200 may assign points based on the concentrations of certain liver proteins and enzymes, prothrombin time (PT) (e.g., clotting time), international normalized ratio (INR) values, and the like. In some embodiments, the drug-disease interaction detection server 200 may assign points for the liver function factor/score according to the levels shown in Table 2 below.

TABLE 2
Liver Function Levels and Corresponding Points Assigned

| Liver Protein or Enzyme | Points Assigned |
|---|---|
| Enzymes - two-fold increase | 1 point |
| Enzymes - three-fold increase | 2 points |
| Alpha Fetoprotein (cancer) - >10 | 1 point |
| Alpha Fetoprotein (cancer) - >400 | 2 points |
| Albumin - >3.5 | 0 points |
| Albumin - 2.8-3.5 | 1 point |
| Albumin - <2.8 | 2 points |
| PT - <4 sec | 0 points |
| PT - 4-6 sec | 1 point |
| PT - >6 sec | 2 points |
| INR - <1.7 | 0 points |
| INR - 1.7-2.3 | 1 point |
| INR - >3 | 2 points |

In some embodiments, the drug-disease interaction detection server 200 may assign points for hemoglobin A1C factor/score according to the levels shown in Table 4 below.

TABLE 3
Hemoglobin A1C Levels and Corresponding Points Assigned

| Hemoglobin A1C Levels | Points Assigned |
|---|---|
| 7-8.0 | 1 points |
| 8.1-9.0 | 2 points |
| 9.1-10+ | 3 points |

In some embodiments, the drug-disease interaction detection server 200 may check the patient's labs and electronic medical record for encephalopathy, by screening the International Statistical Classification of Diseases and related Health Problems (ICD) codes listed in the EMR, and assign 1 point to a corresponding factor in the drug-disease interaction detection score. In some embodiments, the drug-disease interaction detection server 200 may also consider hemoglobin/hematocrit, electrolytes, and urinalysis in the assigning of points based on labs in step 443. In some embodiments, the drug-disease interaction detection server 200 may check pertinent drug levels (e.g., levels of phenytoin, carbamazepine, theophylline, aminoglycosides, and the like) to assess if they are within therapeutic ranges. In some embodiments, if a drug level is not within a therapeutic range, the drug-disease interaction detection server 200 may assign 1 point to a corresponding factor in the drug-disease interaction score and generate an alert indicating that a drug level is out of range.

After assigning points based on the patient's labs, the method 400 proceeds to step 444 in FIG. 4C. At step 444, the drug-disease interaction detection server 200 may determine if the requested medication is contraindicated for any disease states of the patient. In some embodiments, the drug-disease interaction detection server 200 may determine if there is a contraindication with use of the drug after screening the ICD codes in the EMR. If there is a contraindication, then the method 400 in this example proceeds to step 445. At step 445, the drug-disease interaction detection server 200 may generate a notification to the provider in which the medication is flagged as contraindicated and the provider is advised not to prescribe the medication. If there are no contraindications found, then the method 400 in this example proceeds to step 446. At step 446, the drug-disease interaction detection server 200 assigns points to a drug-disease interaction score based on identifying any other disease states of the patient through screening ICD codes listed in the EMR. In some embodiments, drug-disease interaction detection server 200 may identify drug-disease interactions, such as between chronic obstructive pulmonary disease (COPD) and non-selective beta blockers, benign prostatic hyperplasia (BPH) and diuretics or testosterone, Parkinson's and anticholinergics, hypo/hyperthyroidism and warfarin, and the like.

At step 448, the drug-disease interaction detection server 200 may generate a total score for the drug-disease interaction score based on computing a total sum of assigned points for the plurality of factors. In some embodiments, the drug-disease interaction detection server 200 may calculate the total score by computing all of the points for each of the factors assigned in steps 402-446 of the method 400. In some embodiments, each factor may be weighted differently according to a level of criticality or importance of the factor in how it affects drug-disease interactions.

At step 450, the drug-disease interaction detection server 200 may determine whether the total score is less than or greater than or equal to a predetermined threshold. If the total score is less than the predetermined threshold, then the method 400 in this example proceeds to step 452. At step 452, the drug-disease interaction detection server 200 may generate a recommendation indicating a moderate drug-disease interaction for prescribing the treatment requested for the patient. If the total score is greater than or equal to the predetermined threshold, then the method 400 in this example proceeds to step 454. At step 454, the drug-disease interaction detection server 200 may generate a recommendation indicating a major drug-disease interaction for prescribing the treatment requested for the patient. In some embodiments, the recommendation generated in step 452 or step 456 may include a description of the drug-disease interactions and corresponding issues found with the requested treatment for the patient, along with an option for the provider to select a safer alternative treatment.

After generating the recommendation in step 452 or step 454, the method 500 proceeds to step 456. At step 456, the drug-disease interaction detection server 200 may transmit a request for an indication being treated in the patient to provider device 300. In some embodiments, the drug-disease interaction detection server 200 may provide a list of options that the provider may select for the indication being treated in the patient, as well as a section where the provider may submit information.

At step 458, the drug-disease interaction detection server 200 may receive data regarding the indication being treated in the patient from provider device 300. In some embodiments, the drug-disease interaction detection server 200 may receive a selection of an indication from the provider device 300 and/or a description submitted by the provider device 300 regarding the indication and/or one or more symptoms that the patient is experiencing.

At step 460, the drug-disease interaction detection server 200 may determine whether there are alternative treatments available. In some embodiments, the drug-disease interaction detection server 200 may access data from one or more databases 112 to identify safer alternative drugs that may be used to treat the indication. In some embodiments, the drug-disease interaction detection server 200 may run the identified safer alternative drugs through the steps of method 400 to confirm that there are less severe, minimal, or zero drug-disease interactions.

If there alternative treatments are available, then the method 400 in this example proceeds to step 462. At step 462, the drug-disease interaction detection server 200 may generate a recommendation indicating the one or more alternative treatments that are available for the patient. In some embodiments, the recommendation may include drug safety information on each of the alternative treatments, as well as a ranking of the alternative treatments based on how safe the treatment is for the patient.

If there are no alternative treatments available, then the method 400 in this example proceeds to step 464. At step 464, the drug-disease interaction detection server 200 may generate a recommendation indicating that no safer alternative treatments were found for the patient, and notification for a close monitoring recommendation of the patient if prescribing the treatment originally requested for the patient in step 402.

From step 462 or step 464, the method 400 proceeds to step 466. In some embodiments, the drug-disease interaction detection server 200 may receive a selection of a treatment from the provider device 300. In some embodiments, the selection received from the provider device 300 may be the treatment originally requested for the patient in step 402 or one of the alternative treatments selected by the provider from step 462.

At step 468, the drug-disease interaction detection server 200 may transmit an order based on the selection received in step 466 through the EMR system 110. In some embodiments, the drug-disease interaction detection server 200 may transmit an order for the originally requested treatment in step 402 or an order for one of the alternative treatments selected by the provider.

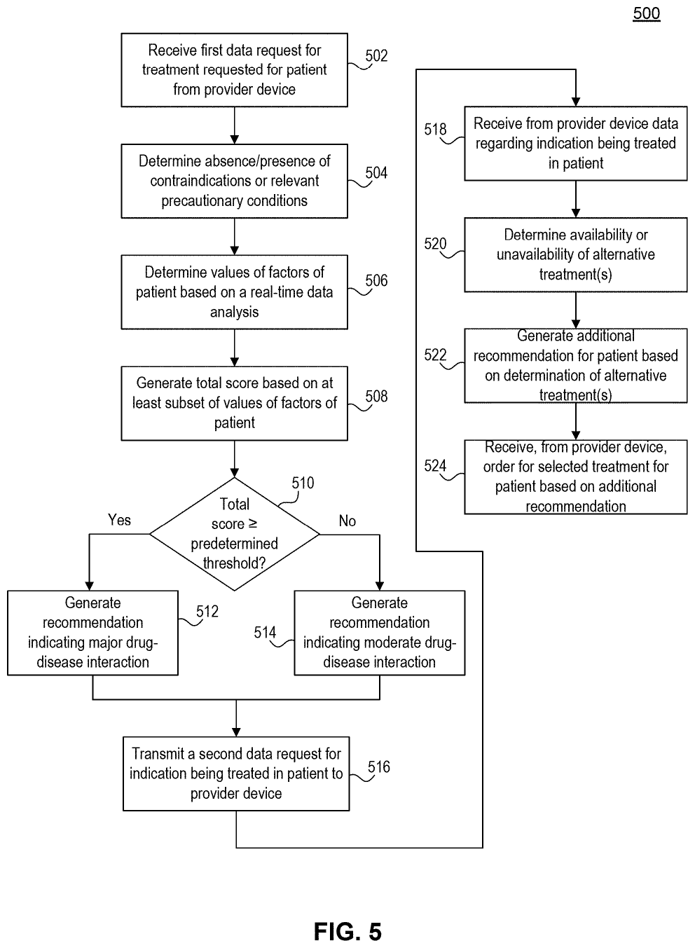

FIG. 5 illustrates an example flowchart diagram of a method 500 for determining drug-disease interactions using a drug-disease interaction detection server, according to embodiments of the present disclosure. The steps of method 500 may be performed by modules of drug-disease interaction detection server 200, such as data collection module 208, pattern recognition module 210, interaction detection module 212, EMR module 214, recommendation module 216, and/or encryption module 218.

Method 500 of FIG. 5 begins with step 502 of receiving a first data request for a treatment request for a patient from a provider device. In some embodiments, drug-disease interaction detection server 200 may receive, from provider device 300, a first data request including data regarding a treatment requested for a patient based on a user input by a provider using provider device 300.

At step 504, the absence or presence of one or more contraindications or relevant precautionary conditions for the treatment may be determined. In some embodiments, drug-disease interaction detection server 200 may determine the absence or presence of one or more contraindications or relevant precautionary conditions for the treatment by parsing at least one database in the plurality of databases 112 based on the treatment. At step 506, values of a plurality of factors corresponding to the patient are determined based on a real-time analysis of data obtained from the EMR system and one or more databases in the plurality of databases. In some embodiments, drug-disease interaction detection server 200 may determine values of a plurality of factors for the patient based on conducting a real-time analysis of data obtained from EMR system 110 and one or more databases 112. In some embodiments, the plurality of factors may include at least one of an age, a gender, or a race of the patient, one or more concurrent drugs and/or herbal medicines used by the patient, one or more genetic variants of the patient, an adverse drug reaction history of the patient, pertinent lab data of the patient, and one or more disease states of the patient.

At step 508, a total score may be generated based on at least a subset of values of the plurality of factors for the patient. In some embodiments, drug-disease interaction detection server 200 may generate a total score by computing a total sum of at least a subset of the values of the plurality of factors corresponding to the patient.

At step 510, it is determined whether the total score is less than or greater than or equal to a predetermined threshold. In some embodiments, drug-disease interaction detection server 200 may determine whether the total score is less than or greater than or equal to a predetermined threshold. If it is determined that the total score is greater than or equal to the predetermined threshold, then method 500 in this example proceeds to step 512. If it is determined that the total score is less than the predetermined threshold, then method 500 in this example proceeds to step 514.

At step 512, in response to determining that the total score is greater than or equal to the predetermined threshold, drug-disease interaction detection server 200 may generate a recommendation indicating a major drug-disease interaction for prescribing the treatment requested for the patient. In some embodiments, a major drug-disease interaction may indicate that prescribing the patient the requested treatment may result in a higher level of severity of adverse drug reactions.

At step 514, in response to determining that the total score is less than the predetermined threshold, drug-disease interaction detection server 200 may generate a recommendation indicating a moderate drug-disease interaction for prescribing the treatment requested for the patient. In some embodiments, a moderate drug-disease interaction may indicate that prescribing the patient the requested treatment may result in a moderate level of severity of adverse drug reactions.

After generating the recommendations in step 512 or step 514, the method 500 proceeds to step 516. At step 516, a second data request is transmitted to the provider device for data regarding the indication being treated in the patient. In some embodiments, drug-disease interaction detection server 200 transmits, to the provider device, a second data request for data regarding the indication being treated in the patient.

At step 518, data regarding the indication being treated in the patient is received. In some embodiments, drug-disease interaction detection server 200 may receive, from the provider device 300, the data regarding the indication being treated in the patient. In some embodiments, the data regarding the indication being treated in the patient may include a description provided by the provider of the patient's diagnosis or condition and/or a description of one or more symptoms that the patient is experiencing.

At step 520, an availability or unavailability of one or more alternative treatments may be determined. In some embodiments, drug-disease interaction detection server 200 may determine an availability or unavailability of one or more alternative treatments to replace the treatment requested for the patient for treating the indication in the patient. In some embodiments, drug-disease interaction detection server 200 may determine if there are one or more safer alternative drugs or medications to prescribe the patient based on the major or moderate drug-disease interactions identified for the requested treatment for the patient.

At step 522, an additional recommendation may be generated for the patient based on the determination of the availability or unavailability of one or more alternative treatments. In some embodiments, drug-disease interaction detection server 200 may generate an additional recommendation for the patient that includes one or more safer alternative drugs or medications to prescribe the patient as identified in step 520. In some embodiments, the additional recommendation generated by the drug-disease interaction detection server 200 may include information indicating a level of drug-disease interactions for each of the one or more safer alternative drugs or medications.

In other embodiments, drug-disease interaction detection server 200 may generate an additional recommendation that includes a notification indicating the unavailability of one or more alternative treatments and a close monitoring recommendation of the patient if prescribing the treatment requested for the patient in the first data request.

At step 524, an order for a selected treatment for the patient may be received from the provider device. In some embodiments, drug-disease interaction detection server 200 may receive, from provider device 300, an order for a selected treatment for the patient based on the additional recommendation, in which the order is transmitted to the EMR system 110. In some embodiments, drug-disease interaction detection server 200 may receive an order for an alternative treatment if alternative treatments are available according to the determination in step 520. In some embodiments, drug-disease interaction detection server 200 may receive an order for the treatment originally requested for the patient in the first data request in step 502, if alternative treatments are unavailable according to the determination in step 520. In some embodiments, the treatment originally requested for the patient in the first data request may be ordered if the drug-disease interactions are determined to be moderate in step 514.

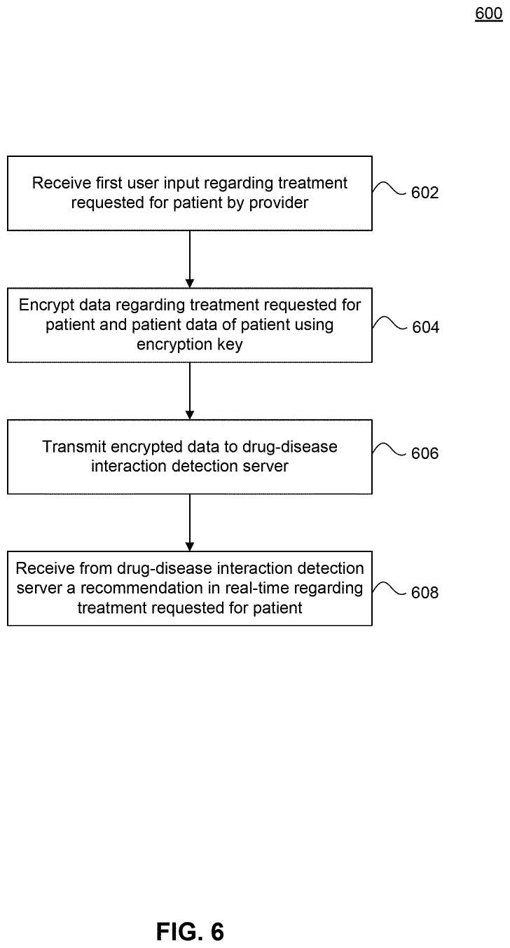

FIG. 6 illustrates an example flowchart diagram of a method 600 for encrypting data and receiving recommendations regarding drug-disease interactions at a provider device, according to embodiments of the present disclosure. The steps of method 600 may be performed by provider device 300. Method 600 of FIG. 6 begins with step 602 of receiving a first user input regarding a treatment requested for a patient by a provider. In some embodiments, a provider may submit a data request using drug-disease interaction detection application 312 and I/O devices 304 of provider device 300, in which the data request includes a name of the requested treatment.

At step 604, data regarding the treatment requested for the patient and patient data of the patient may be encrypted using an encryption key. In some embodiments, encryption module 313 may encrypt the data regarding the treatment and patient data using one or more encryption keys. In some embodiments, the patient data may include at least one of a patient name, an identification number, a medical record number, a date of birth, or a phone number of the patient.

At step 606, the encrypted data may be transmitted to a drug-disease interaction detection server. In some embodiments, drug-disease interaction detection application 312 and/or encryption module 313 may transmit the encrypted data via network interface 308 to drug-disease interaction detection server 200 over network 104. In some embodiments, drug-disease interaction detection server 200 may be configured to decrypt the encrypted data upon receipt.

At step 608, a recommendation regarding the treatment requested for the patient may be received from the drug-disease interaction detection server in real-time. In some embodiments, drug-disease interaction detection application 312 may receive a recommendation in real-time regarding the treatment requested for the patient from the drug-disease interaction detection server 200. In some embodiments, the recommendation received at the drug-disease interaction detection application 312 may include information identifying one or more drug-disease interactions of the treatment requested for the patient.

In some embodiments, the recommendation may include one or more alternative treatments to prescribe the patient based on the information identifying the one or more drug-disease interactions of the treatment requested for the patient. In some embodiments, the recommendation may include one or more options for the provider to provide additional user input regarding an indication associated with the treatment requested for the patient.

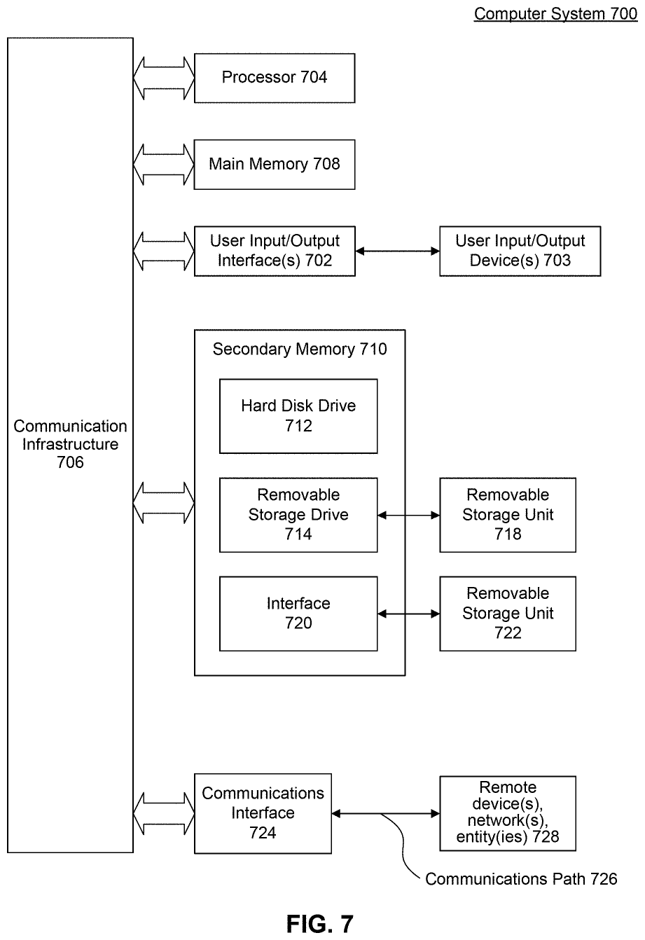

FIG. 7 is a block diagram of example components of computer system 700. One or more computer systems 700 may be used, for example, to implement any of the embodiments discussed herein, as well as combinations and sub-combinations thereof. In some embodiments, one or more computer systems 700 may be used to implement the methods 400, 500, and 600 shown in FIGS. 4A-6, respectively, drug-disease interaction detection server 106, 200, provider device 102, 300, and EMR system 110 shown in FIGS. 1-3, as described herein. Computer system 700 may include one or more processors (also called central processing units, or CPUs), such as a processor 704. Processor 704 may be connected to a communication infrastructure or bus 706.

Computer system 700 may also include user input/output interface(s) 702, such as monitors, keyboards, pointing devices, etc., which may communicate with communication infrastructure 706 through user input/output device(s) 703.

One or more of processors 704 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 700 may also include a main or primary memory 708, such as random access memory (RAM). Main memory 708 may include one or more levels of cache. Main memory 708 may have stored therein control logic (i.e., computer software) and/or data. In some embodiments, main memory 708 may include optical logic configured to perform real-time drug-disease state interaction detection and generate recommendations

Computer system 700 may also include one or more secondary storage devices or memory 710. Secondary memory 710 may include, for example, a hard disk drive 712 and/or a removable storage drive 714.

Removable storage drive 714 may interact with a removable storage unit 718.

Removable storage unit 718 may include a computer usable or readable storage device having stored thereon computer software (control logic) and/or data. Removable storage unit 718 may be a program cartridge and cartridge interface (such as that found in video game devices), a removable memory chip (such as an EPROM or PROM) and associated socket, a memory stick and USB port, a memory card and associated memory card slot, and/or any other removable storage unit and associated interface. Removable storage drive 714 may read from and/or write to removable storage unit 718.

Secondary memory 710 may include other means, devices, components, instrumentalities or other approaches for allowing computer programs and/or other instructions and/or data to be accessed by computer system 700. Such means, devices, components, instrumentalities or other approaches may include, for example, a removable storage unit 722 and an interface 720. Examples of the removable storage unit 722 and the interface 720 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 700 may further include a communication or network interface 724. Communication interface 724 may enable computer system 700 to communicate and interact with any combination of external devices, external networks, external entities, etc. (individually and collectively referenced by reference number 728). For example, communication interface 724 may allow computer system 700 to communicate with external or remote devices 728 over communications path 726, which may be wired and/or wireless (or a combination thereof), and which may include any combination of LANs, WANs, the Internet, etc. Control logic and/or data may be transmitted to and from computer system 700 via communication path 726.

Computer system 700 may also be any of a personal digital assistant (PDA), desktop workstation, laptop or notebook computer, netbook, tablet, smartphone, smartwatch or other wearables, appliance, part of the Internet-of-Things, and/or embedded system, to name a few non-limiting examples, or any combination thereof

Computer system 700 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 700 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 700, main memory 708, secondary memory 710, and removable storage units 718 and 722, as well as tangible articles of manufacture embodying any combination of the foregoing. Such control logic, when executed by one or more data processing devices (such as computer system 700), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 7. In particular, embodiments can operate with software, hardware, and/or operating system implementations other than those described herein.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

Embodiments of the present disclosure have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. A computer-implemented method comprising:
receiving, at a computing platform and from a provider device of a provider, a first data request comprising data regarding a treatment requested for a patient based on a user input by the provider, wherein the computing platform, the provider device, an electronic medical records (EMR) system, and a plurality of databases are communicatively coupled via a network;
determining, by at least one processor of the computing platform, a presence or absence of one or more contraindications or relevant precautionary conditions for the treatment by parsing at least one database in the plurality of databases based on the treatment;
upon determining the presence or absence of one or more contraindications or relevant precautionary conditions, determining values of a plurality of factors corresponding to the patient based on a real-time analysis of data obtained from the EMR system and one or more databases in the plurality of databases, wherein the plurality of factors comprise at least one of an age, a gender, or a race of the patient, one or more concurrent drugs and/or herbal medicines used by the patient, one or more genetic variants of the patient, an adverse drug reaction history of the patient, pertinent lab data of the patient, and one or more disease states of the patient;
generating a total score by computing a total sum of at least a subset of the values of the plurality of factors corresponding to the patient;
determining whether the total score is less than or greater than or equal to a predetermined threshold;
generating a recommendation for prescribing the treatment requested for the patient in response to the determination of the total score and an indication being treated in the patient, the generating comprising:
in response to determining that the total score is less than the predetermined threshold, generating a recommendation indicating a moderate drug-disease interaction for prescribing the treatment requested for the patient; and
in response to determining that the total score is greater than or equal to the predetermined threshold, generating a recommendation indicating a major drug-disease interaction for prescribing the treatment requested for the patient;
in response to the recommendation, transmitting, to the provider device, a second data request for data regarding the indication being treated in the patient;
receiving, from the provider device, the data regarding the indication being treated in the patient;
determining, by the at least one processor, an availability or unavailability of one or more alternative treatments to replace the treatment requested for the patient for treating the indication in the patient;
generating an additional recommendation for the patient based on the determination of the availability or the unavailability of the one or more alternative treatments; and
receiving, from the provider device, an order for a selected treatment for the patient based on the additional recommendation, wherein the order is transmitted to the EMR system.

2. The computer-implemented method of claim 1, wherein the data regarding the treatment requested for the patient in the first data request received from the provider device is encrypted, the method further comprising:
decrypting, by the at least one processor, the data regarding the treatment requested for the patient using an encryption key.

3. The computer-implemented method of claim 1, wherein the data regarding the indication being treated in the patient received from the provider device is encrypted, the method further comprising:
decrypting, by the at least one processor, the data regarding the indication being treated in the patient using an encryption key.

4. The computer-implemented method of claim 1, wherein generating the additional recommendation for the patient based on the determination of the availability or the unavailability of the one or more alternative treatments comprises:
generating the additional recommendation comprising a notification indicating the unavailability of the one or more alternative treatments and a close monitoring recommendation of the patient if prescribing the treatment requested for the patient in the first data request.

5. The computer-implemented method of claim 1, wherein generating the additional recommendation for the patient based on the determination of the availability or the unavailability of the one or more alternative treatments comprises:
generating the additional recommendation comprising a notification indicating the availability of the one or more alternative treatments, data regarding the one or more alternative treatments, and information indicating a level of drug-disease interactions for each of the alternative treatments.

6. The computer-implemented method of claim 1, wherein data regarding the one or more concurrent drugs and/or herbal medicines used by the patient further comprises data regarding kinetic, dynamic, and pharmacogenomics interactions from the one or more concurrent drugs and/or herbal medicines.

7. The computer-implemented method of claim 1, wherein the plurality of databases comprises a drug-disease interaction database, the method further comprising:
generating, by the at least one processor, a catalog of a plurality of medications comprising information regarding at least one of kinetic, dynamic, or pharmacogenomic interactions for each medication based on drug manufacturing information, medical literature, and established medical guidelines; and
updating the drug-disease interaction database based on the catalog of the plurality of medications.

8. The computer-implemented method of claim 7, the method further comprising:
updating the drug-disease interaction database further based on scientific literature data and historical system data using one or more machine learning algorithms.

9. A drug-disease interaction detection server comprising:
a plurality of databases; and
at least one processor, wherein the drug-disease interaction detection server is communicatively coupled to a provider device and an electronic medical records (EMR) system via a network, and wherein the at least one processor is configured to:
receive, from the provider device of a provider, a first data request comprising data regarding a treatment requested for a patient based on a user input by the provider;
determine a presence or absence of one or more contraindications or relevant precautionary conditions for the treatment by parsing at least one database in the plurality of databases based on the treatment;
upon determining the presence or absence of one or more contraindications or relevant precautionary conditions, determine values of a plurality of factors corresponding to the patient based on a real-time analysis of data obtained from the EMR system and one or more databases in the plurality of databases, wherein the plurality of factors comprise at least one of an age, a gender, or a race of the patient, one or more concurrent drugs and/or herbal medicines used by the patient, one or more genetic variants of the patient, an adverse drug reaction history of the patient, pertinent lab data of the patient, and one or more disease states of the patient;
generate a total score by computing a total sum of at least a subset of the values of the plurality of factors corresponding to the patient;
determine whether the total score is less than or greater than or equal to a predetermined threshold;
generate a recommendation for prescribing the treatment requested for the patient in response to the determination of the total score and an indication being treated in the patient, the generating comprising:
in response to determining that the total score is less than the predetermined threshold, generating a recommendation indicating a moderate drug-disease interaction for prescribing the treatment requested for the patient; and
in response to determining that the total score is greater than or equal to the predetermined threshold, generating a recommendation indicating a major drug-disease interaction for prescribing the treatment requested for the patient;
in response to the recommendation, transmit, to the provider device, a second data request for data regarding the indication being treated in the patient;
receive, from the provider device, the data regarding the indication being treated in the patient;
determine an availability or unavailability of one or more alternative treatments to replace the treatment requested for the patient for treating the indication in the patient;
generate an additional recommendation for the patient based on the determination of the availability or the unavailability of the one or more alternative treatments; and
receive, from the provider device, an order for a selected treatment for the patient based on the additional recommendation, wherein the order is transmitted to the EMR system.

10. The drug-disease interaction detection server of claim 9, wherein the data regarding the treatment requested for the patient in the first data request received from the provider device is encrypted, and wherein the at least one processor is further configured to:
decrypt the data regarding the treatment requested for the patient using an encryption key.

11. The drug-disease interaction detection server of claim 9, wherein the data regarding the indication being treated in the patient received from the provider device is encrypted, and wherein the at least one processor is further configured to:
decrypt the data regarding the indication being treated in the patient using an encryption key.

12. The drug-disease interaction detection server of claim 9, wherein generating the additional recommendation for the patient based on the determination of the availability or the unavailability of the one or more alternative treatments comprises:
generating the additional recommendation comprising a notification indicating the unavailability of the one or more alternative treatments and a close monitoring recommendation of the patient if prescribing the treatment requested for the patient in the first data request.

13. The drug-disease interaction detection server of claim 9, wherein generating the additional recommendation for the patient based on the determination of the availability or the unavailability of the one or more alternative treatments comprises:
generating the additional recommendation comprising a notification indicating the availability of the one or more alternative treatments, data regarding the one or more alternative treatments, and information indicating a level of drug-disease interactions for each of the alternative treatments.

14. The drug-disease interaction detection server of claim 9, wherein the plurality of databases comprises a drug-disease interaction database, and wherein the at least one processor is further configured to:
generate a catalog of a plurality of medications comprising information regarding at least one of kinetic, dynamic, or pharmacogenomic interactions for each medication based on drug manufacturing information, medical literature, and established medical guidelines; and
update the drug-disease interaction database based on the catalog of the plurality of medications.

15. A provider device comprising:
a memory;
a network interface; and
at least one processor coupled to the memory, wherein the provider device is communicatively coupled to a drug-disease interaction detection server and an electronic medical records (EMR) system via the network interface, and wherein the at least one processor is configured to:
receive a first user input comprising data regarding a treatment requested for a patient by a provider;
encrypt the data regarding the treatment requested for the patient and patient data comprising a patient name and identification information of the patient using an encryption key, resulting in encrypted data; and
transmit the encrypted data to the drug-disease interaction detection server; and
in response to transmitting the encrypted data, receive, from the drug-disease interaction detection server, a recommendation in real-time regarding the treatment requested for the patient, wherein the recommendation provides information identifying one or more drug-disease interactions of the treatment requested for the patient.

16. The provider device of claim 15, wherein the recommendation further comprises one or more alternative treatments to prescribe the patient based on the information identifying the one or more drug-disease interactions of the treatment requested for the patient.

17. The provider device of claim 15, wherein the recommendation further comprises one or more options for the provider to provide additional user input regarding an indication associated with the treatment requested for the patient.

18. The provider device of claim 17, wherein the at least one processor is further configured to:
in response to the one or more options in the recommendation, receive a second user input comprising data regarding the indication associated with the treatment requested for the patient;
encrypt the data regarding the indication associated with the treatment requested for the patient, resulting in encrypted indication data;
transmit the encrypted indication data to the drug-disease interaction detection server; and
receive, from the drug-disease interaction detection server, one or more alternative treatments to prescribe the patient based on the indication associated with the treatment requested for the patient.

19. The provider device of claim 18, wherein the at least one processor is further configured to:
receive a third user input comprising a selection of one of the one or more alternative treatments by the provider; and
generate an order based on the selection of one of the one or more alternative treatments; and
transmit the order to the EMR system.

20. The provider device of claim 15, wherein the identification information of the patient comprises at least one of an identification number, a medical record number, a date of birth, or a phone number of the patient.
